(19) United States
(12) Patent Application Publication
(10) Pub. No.: US 2026/0224826 A1
(43) Pub. Date: Aug. 6, 2026

(54) Flexible Supine Wearable Pelvic Floor Drug Nebulization Device

(71) Applicants: Jing Lu, Rancho Cucamonga, CA (US); Yong Zhang, Xi'an (CN); Jie Shao, Xi'an (CN); Zicai Zhu, Xi'an (CN)

(72) Inventors: Jing Lu, Rancho Cucamonga, CA (US); Yong Zhang, Xi'an (CN); Jie Shao, Xi'an (CN); Zicai Zhu, Xi'an (CN)

(21) Appl. No.: 19/046,567

(22) Filed: Feb. 6, 2025

(51) Int. Cl. A61M 11/00; A61M 11/02

(52) U.S. Cl. CPC A61M 11/005; A61M 11/02; A61M 2210/1067

(57) ABSTRACT

This application presents a flexible supine wearable pelvic floor drug nebulization device, comprising a wearable nebulization hood and a nebulizer. The hood features a body with drainage and exhaust ports, an open end, and a nebulization outlet connected to the nebulizer. The device is secured around the waist with a waist tightening belt. It also includes an expandable nebulization membrane designed to cover the anus and surrounding areas. The membrane's curved side edges match the contours of the buttocks, with the edges sealed by sealing gel. This device is intended for post-operative wound disinfection and drug nebulization treatments, particularly for areas such as the anus. Compared to traditional drug wiping methods, it offers enhanced comfort, more precise drug delivery, better absorption efficiency, and minimizes patient discomfort.

### TECHNICAL FIELD

The present application relates to the field of medical devices, particularly to a flexible supine wearable pelvic floor drug nebulization device.

### BACKGROUND

After anal surgery, patients typically face issues such as slow wound healing, local edema, and inflammation, which adversely affect their recovery. Traditional treatment methods, such as oral medication, topical ointments, or injections, may alleviate symptoms to some extent, but they generally suffer from problems such as incomplete drug absorption, imprecise local action, and poor patient compliance. This is especially true in the case of the anus, where traditional local drug delivery methods often fail to achieve optimal therapeutic results.

Currently, drug nebulization technology, as a non-invasive, non-invasive method of local drug delivery, has been widely used in the treatment of respiratory diseases. Existing drug nebulization devices are mainly designed for nebulizing medications for the respiratory system. Although these devices have shown some effectiveness in respiratory treatments, their design has not taken into account the special needs of the surgical area after anal surgery, and they suffer from problems such as inaccurate control of nebulization particle size, uneven spray, and low drug delivery efficiency.

Furthermore, existing nebulization devices are generally universal and lack specialized design for the treatment of the anal region. They are unable to accurately control the direction and volume of drug spraying, resulting in unsatisfactory therapeutic effects. During use, patients often face difficulties operating the devices, and they must lie flat during treatment to reduce wound tension. Therefore, there is an urgent need for a device specifically designed for drug nebulization treatment after anal surgery, capable of accurately delivering medication, improving treatment efficacy, and enhancing patient compliance.

### BRIEF SUMMARY

The embodiment of the present application provides a flexible supine wearable pelvic floor drug nebulization device, which can be used for post-operative wound disinfection and drug nebulization treatment for areas such as the anus. Compared to traditional drug wiping methods, this device offers greater comfort, more precise drug delivery, higher absorption efficiency, and reduces patient discomfort. Furthermore, the device is designed for use in a supine position, adapts to the contours of the buttocks and legs, provides comfortable treatment, and avoids tension at the wound site to prevent further injury.

In order to address the above technical problems, the embodiment of the present application provides a flexible supine wearable pelvic floor drug nebulization device, comprising: a wearable nebulization hood and a nebulizer; the wearable nebulization hood includes a hood body and drainage and exhaust openings arranged on the hood body; one end of the hood body is open, and the other end is provided with a nebulization outlet connected to the nebulizer; a waist tightening belt is provided at the opening of the hood body to secure the wearable nebulization hood around the waist; the hood body further includes an expandable nebulization membrane for covering the anus and surrounding areas; the side edges of the expandable nebulization membrane are curved, with the curvature of the side edges matching the contour of the human buttocks, and the edges of the curved side are sealed with sealing gel.

In some exemplary embodiments, the waist tightening belt is equipped with an adjustable adhesive waistband; during use, the nebulization device is worn on the lower part of the body, and the waist tightening belt is used to secure the wearable nebulization hood around the waist, with the adjustable adhesive waistband used for further fixing and adjusting; the adjustable adhesive waistband includes a first adjustable adhesive waistband and a second adjustable adhesive waistband, with the first and second adjustable adhesive waistbands located on the left and right sides of the waist, respectively.

In some exemplary embodiments, the nebulization outlet is located at the bottom of the wearable nebulization hood, directly below the expandable nebulization membrane.

In some exemplary embodiments, the flexible supine wearable pelvic floor drug nebulization device further comprises: a drainage bag; the drainage bag is connected to the drainage opening of the wearable nebulization hood.

In some exemplary embodiments, the drainage opening is connected to the inlet of the drainage bag, and the drainage opening is located at the lower part of the wearable nebulization hood, used for discharging the residual medication from the condensation of the nebulized mist.

In some exemplary embodiments, the expandable nebulization membrane is made of a film material, and the nebulized spray is blown into the expandable nebulization membrane through the nebulization outlet. Positive pressure is generated within the expandable nebulization membrane, causing it to expand and adapt to the contour of the buttocks and legs, ensuring comfortable wear.

In some exemplary embodiments, the sealing gel is made of a silicone gel material with antibacterial properties, waterproof, air-tight, soft, adhesive, and non-damaging to the skin.

In some exemplary embodiments, the flexible supine wearable pelvic floor drug nebulization device further comprises: a spraying device; the spraying device is connected to the nebulization outlet to clean the anal area.

In some exemplary embodiments, the nebulizer uses a compressed air nebulizer or a micro-mesh ultrasonic nebulizer, and the output port of the nebulizer is connected to the nebulization outlet of the wearable nebulization hood through a pipeline.

In some exemplary embodiments, the output port is a one-way exhaust outlet.

The technical solution provided by the embodiment of this application has at least the following advantages:

The technical solution provided by the embodiment of the present application has at least the following advantages: The flexible supine wearable pelvic floor drug nebulization device includes: a wearable nebulization hood and a nebulizer; the wearable nebulization hood includes a hood body and drainage and exhaust openings arranged on the hood body; one end of the hood body is open, and the other end is provided with a nebulization outlet connected to the nebulizer; a waist tightening belt is provided at the opening of the hood body to secure the wearable nebulization hood around the waist; the hood body further includes an expandable nebulization membrane for covering the anus and surrounding areas; the side edges of the expandable nebulization membrane are curved, with the curvature of the side edges matching the contour of the human buttocks, and the edges of the curved side are sealed with sealing gel.

The flexible supine wearable pelvic floor drug nebulization device provided by the embodiment of the present application has the following beneficial effects compared to the prior art:
1. More precise drug delivery: Drug nebulization can convert the drug into fine mist droplets, ensuring that the drug evenly covers the entire anal area and wound surface, directly acting on the wound. In contrast, the drug wiping method may fail to effectively cover the area due to uneven application or improper pressure.
2. Increased drug absorption efficiency: Drug nebulization directly acts on the localized area, where the drug contacts the affected area, increasing the local drug concentration. This accelerates wound healing, reduces edema, and alleviates inflammation. In comparison, wiping may result in lower absorption and effectiveness due to the drug's attachment to the external surface or limited wound cleanliness.
3. Reduced patient discomfort: Drug wiping typically requires manual operation by the patient and may cause discomfort or pain, especially in the sensitive anal area. Drug nebulization, as a non-invasive treatment method, does not require direct contact with the wound and avoids additional physical irritation, thereby increasing patient comfort and improving patient compliance.
4. Avoidance of wound tearing due to tension: The device of the present invention is used in a supine position, avoiding the tension that can cause tearing of the wound during nebulization while sitting or squatting. Additionally, the device is flexible and expandable, adapting to the shape of the buttocks and legs, and uses silicone gel sealing to reduce skin damage and enhance comfort.

### BRIEF DESCRIPTION OF DRAWINGS

One or more embodiments are illustrated in the accompanying drawings, which are provided for illustrative purposes and do not limit the embodiments, unless specifically stated. The figures in the drawings do not impose proportional limitations.

FIG. 1 is an overall schematic view showing the use of the flexible supine wearable pelvic floor drug nebulization device according to an embodiment of the present application.

FIG. 2 is a side view of the wearable nebulizer hood according to an embodiment of the present application.

FIG. 3 is a front view of the wearable nebulizer hood according to an embodiment of the present application.

FIG. 4 is a bottom structure view of the wearable nebulizer hood according to an embodiment of the present application.

FIG. 5 is a three-dimensional schematic view of the wearable nebulizer hood according to an embodiment of the present application.

### REFERENCE NUMERALS

1—Wearable nebulizer hood, 2—nebulizer, 3—drainage bag, 11—waist tightening belt, 12—sealing gel, 13—expandable nebulization membrane, 14—drainage port, 15—adjustable waist adhesive tape, 16—exhaust hole, 17—nebulization port, 151—first adjustable waist adhesive tape, 152—second adjustable waist adhesive tape.

### DETAILED DESCRIPTION

As can be seen from the background art, the design of existing nebulization devices does not take into account the specific requirements of the surgical area after anal surgery. There are problems such as inaccurate control of the nebulization particle size, uneven spray, and low drug delivery efficiency.

To solve the above technical problems, the embodiment of this application provides a flexible supine wearable pelvic floor drug nebulization device, including: a wearable nebulizer hood and a nebulizer. The wearable nebulizer hood includes a hood body, and drainage and exhaust ports set on the hood body. One end of the hood body is open, and the other end is provided with a nebulization port, which is connected to the nebulizer. The opening of the hood body is equipped with a waist tightening belt to secure the wearable nebulizer hood to the waist. The hood body also has an expandable nebulization membrane for wrapping the anus and surrounding area. The side of the expandable nebulization membrane is arcuate, and the curvature of the side edge matches the curvature of the human buttocks, and is sealed with sealing gel along the side edge.

The flexible supine wearable pelvic floor drug nebulization device provided in this application can be used for the disinfection and drug nebulization treatment of postoperative wounds, such as those in the anal area. Compared to traditional drug wiping, this device is more comfortable to use, provides more precise drug delivery, has higher absorption efficiency, and reduces patient discomfort. Additionally, the device can be used in a supine position, adapts to the shape of the buttocks and legs, and is comfortable for treatment while avoiding tension on the wound that could cause re-injury.

The following detailed description will explain the embodiments of the present application with reference to the drawings. However, those skilled in the art will understand that the technical details presented are for the purpose of helping the reader better understand this application. Even without these technical details and the various changes and modifications based on the embodiments described below, the technical solution claimed in this application can still be realized.

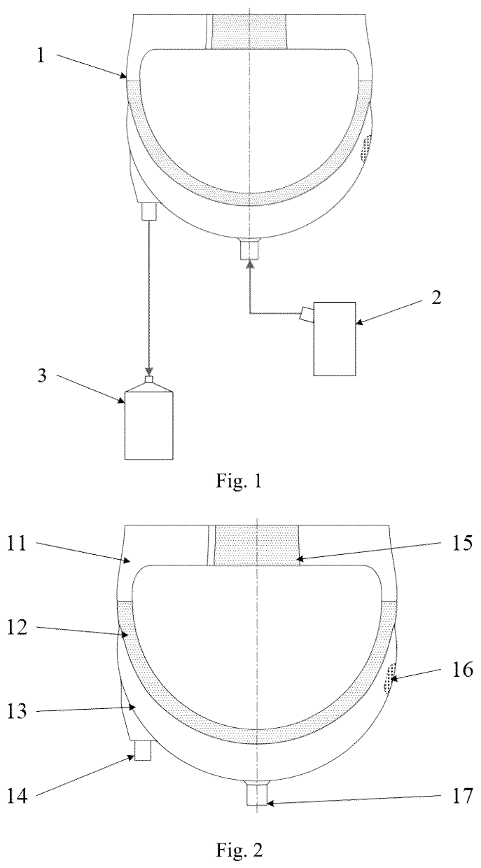

With reference to FIG. 1 and FIG. 2, the embodiment of this application provides a flexible supine wearable pelvic floor drug nebulization device, which includes: a wearable nebulizer hood 1 and a nebulizer (2). The wearable nebulizer hood (1) includes a hood body, with a drainage port (14) and an exhaust port (16) mounted on the hood body. One end of the hood body is open, and the other end is provided with a nebulization port (17), which is connected to the nebulizer (2). The opening of the hood body is equipped with a waist tightening belt (11) to secure the wearable nebulizer hood (1) to the waist. The hood body also includes an expandable nebulization membrane (13) for covering the anus and surrounding area. The side of the expandable nebulization membrane (13) is arcuate, with the curvature of the side edge matching the curvature of the human buttocks, and it is sealed along the edge with sealing gel (12).

The flexible supine wearable pelvic floor drug nebulization device provided in this application is worn on the lower part of the human body. The waist tightening belt (11) is fastened around the waist, and the tightness can be adjusted using an adjustable waist adhesive tape (15). The top opening of the expandable nebulization membrane (13) is connected to the waist tightening belt (11), and the expandable nebulization membrane (13) is used to wrap the anus and surrounding area and is sealed with sealing gel (12). The drainage port (14) of the wearable nebulizer hood (1) can be connected to the drainage bag (3), and the nebulization port (17) is connected to the nebulizer (2).

It should be noted that the expandable nebulization membrane (13) of the wearable nebulizer hood (1) is in the form of a film that, during nebulization, expands to adapt to the contours of the pelvic floor and legs, providing comfortable wear.

In some embodiments, the waist tightening belt (11) is equipped with an adjustable adhesive strip (15) on the waist. When in use, the nebulization device is worn on the lower part of the human body. The waist tightening belt (11) is fastened around the waist, and the adjustable adhesive strip (15) is used to fix and adjust the tightness. The adjustable adhesive strip (15) includes a first adjustable adhesive strip (151) and a second adjustable adhesive strip (152), located on the left and right sides of the waist, respectively.

In some embodiments, the nebulization port (17) is positioned at the bottom of the wearable nebulizer hood (1) and directly beneath the expandable nebulization membrane (13).

In some embodiments, the flexible supine wearable pelvic floor drug nebulization device further includes a drainage bag (3). The drainage bag (3) is connected to the drainage port (14) of the wearable nebulizer hood (1).

In some embodiments, the drainage port (14) is connected to the intake port of the drainage bag (3), and the drainage port (14) is positioned at the lower part of the wearable nebulizer hood (1). This means that the drainage port (14) is positioned lower than the wearable nebulizer hood (1), and is used to discharge the residual medication from the condensed mist after nebulization.

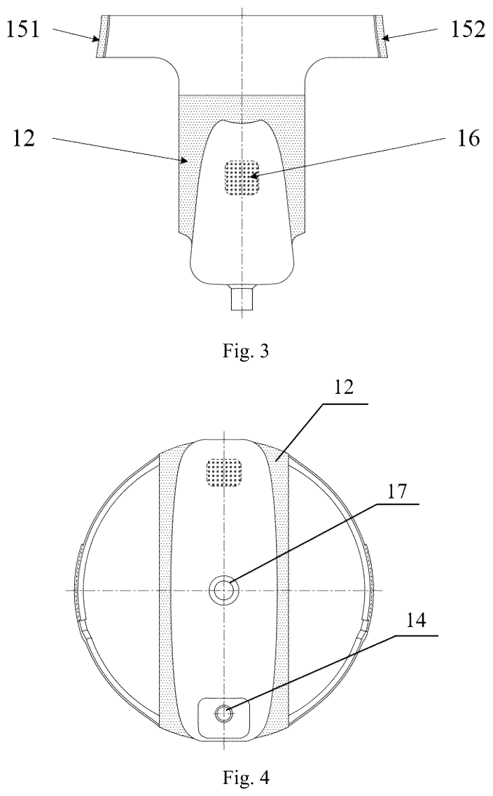

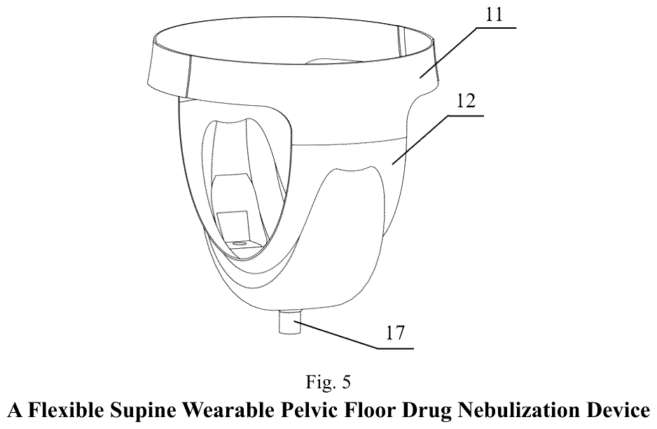

As shown in FIGS. 1-5, one embodiment of the present application provides a flexible supine wearable pelvic floor drug nebulization device, which includes: a wearable nebulizer hood (1), a nebulizer (2), and a drainage bag (3). The wearable nebulizer hood (1) consists of a waist tightening belt (11), sealing gel (12), an expandable nebulization membrane (13), a drainage port (14), an adjustable waist adhesive strip (15), an exhaust port (16), and a nebulization port (17). When in use, the wearable nebulizer hood (1) is worn on the lower part of the body, with the waist tightening belt (11) fastened around the waist. The tightness is adjusted using the adjustable waist adhesive strip (15). The expandable nebulization membrane (13) wraps the anus and surrounding area, and is sealed with sealing gel (12). The drainage port (14) of the wearable nebulizer hood (1) is connected to the drainage bag (3), and the nebulization port (17) is connected to the nebulizer (2).

In some embodiments, the expandable nebulization membrane (13) is made from a thin film material. The nebulized mist is directed into the expandable nebulization membrane (13) through the nebulization port (17), creating positive pressure inside the membrane, which causes it to expand and adapt to the contours of the buttocks and legs, providing a comfortable fit.

In some embodiments, the sealing gel (12) is made from a silicone gel material, which is antibacterial, waterproof, leak-proof, soft, adhesive, and does not harm the skin.

In some embodiments, the flexible supine wearable pelvic floor drug nebulization device further includes a spraying device. The spraying device is connected to the nebulization port (17) for cleaning the anal area. In this case, the nebulization port (17) of the wearable nebulizer hood (1) serves as a cleaning port, which is connected to the spraying device for cleaning the anal area.

In some embodiments, the nebulizer (2) is a compressed air nebulizer or a micro-mesh ultrasonic nebulizer. The output port of the nebulizer (2) is connected to the nebulization port (17) of the wearable nebulizer hood (1) via a pipeline.

In some embodiments, the exhaust port (16) serves as the exhaust outlet for the wearable nebulizer hood (1) and is a unidirectional exhaust vent.

Specifically, the exhaust outlet of the wearable nebulizer hood (1) is unidirectional and opens under a certain pressure to ensure that positive pressure is maintained inside the expandable nebulization membrane (13), allowing it to expand and adapt to the contours of the buttocks and legs.

It should be noted that the wearable nebulizer hood (1) and the drainage bag (3) are low-cost and can be made for single-use, improving safety, while the nebulizer (2) is reusable.

The flexible supine wearable pelvic floor drug nebulization device provided by this embodiment operates as follows:
1. During use, the wearable nebulizer hood (1) is worn on the lower part of the body. The waist tightening belt (11) is fastened around the waist, and the adjustable adhesive strip (15) is used to secure it. The expandable nebulization membrane (13) wraps around the anus and surrounding area and is sealed with sealing gel (12) to prevent water and air leakage. The drainage port (14) of the wearable nebulizer hood (1) is connected to the drainage bag (3), and the nebulization port (17) is connected to the nebulizer (2).
2. The medicinal solution is added to the nebulizer (2), which is then activated to produce mist. The nebulized mist is blown into the expandable nebulization membrane (13) through the nebulization port (17). Because the exhaust port (16) must open under a certain pressure, a slight positive pressure is formed within the expandable nebulization membrane (13), causing it to expand and adapt to the contours of the buttocks and legs, ensuring comfortable wear.
3. During nebulization, the medicinal mist disinfects the wound, with the mist evenly covering the anal area and wound surface. This promotes wound healing, reduces swelling, and alleviates inflammation. The condensed residual medicinal solution is expelled through the drainage port (14) of the wearable nebulizer hood (1).
4. After nebulization, the wearable nebulizer hood (1) is designed for single-use, preventing cross-contamination from reuse. The nebulizer (2) can be detached, cleaned, and reused.

The flexible supine wearable pelvic floor drug nebulization device provided in this embodiment includes: a wearable nebulizer hood (1) and a nebulizer (2). The wearable nebulizer hood (1) comprises a hood body, drainage port (14), and exhaust port (16). One end of the hood body is open, and the other end is fitted with a nebulization port (17), which is connected to the nebulizer (2). The open end of the hood body is equipped with a waist tightening belt (11) for securing the device around the waist. The hood body also includes an expandable nebulization membrane (13) that wraps around the anus and surrounding area. The edges of the expandable nebulization membrane (13) are arc-shaped, and the arc is adapted to the curvature of the human buttocks. The edges are sealed with sealing gel (12).

The flexible supine wearable pelvic floor drug nebulization device provided by this application offers the following advantages over the prior art:
1. More precise drug delivery: Nebulized drug delivery can transform the medication into fine droplets, ensuring uniform coverage of the anal region and wound surface, allowing the medication to directly act on the wound. In contrast, manual application methods may result in uneven coverage or ineffective application due to improper pressure or technique.
2. Improved drug absorption efficiency: Nebulized medication directly targets the affected area, leading to higher localized drug concentrations, which accelerates wound healing, reduces swelling, and alleviates inflammation. In contrast, wiping the anal area may result in poor drug absorption and limited effectiveness due to surface attachment or inadequate cleanliness.
3. Reduced patient discomfort: Drug wiping typically requires manual intervention from the patient, which may cause discomfort or pain, especially in sensitive areas like the anus. Nebulized drug delivery, being non-invasive, does not require direct contact with the wound, avoiding additional physical irritation. This improves patient comfort and compliance.
4. Prevention of wound tension and tears: The device is designed for use in a supine position, which prevents the tension on the wound caused by squatting or sitting during traditional cleaning or nebulization. The flexible, expandable design of the device adapts to the contours of the buttocks and legs, reducing skin damage and increasing comfort, thanks to the use of silicone gel for sealing.

Those skilled in the art will understand that the embodiments described above are specific examples and that various changes and modifications can be made to them without departing from the spirit and scope of the application. Any modifications or variations that fall within the scope of the claims are intended to be encompassed by the present application. The scope of protection should therefore be determined by the claims.

1. A flexible supine wearable pelvic floor drug nebulization device, comprising: a wearable nebulization hood (1) and a nebulizer (2).
The wearable nebulization hood (1) comprising a hood body with a drainage port (14) and an exhaust port (16) disposed on the hood body. One end of the hood body is open, and the other end is provided with a nebulization port (17), with the nebulization port (17) connected to the nebulizer (2).
The open end of the hood body being equipped with a waist tightening belt (11) to secure the wearable nebulization hood (1) to the waist.
The hood body further comprising an expandable nebulization membrane (13) to wrap around the anus and surrounding area, with the side edges of the expandable nebulization membrane (13) being arc-shaped, the curvature of the side edges matching the curvature of the human buttocks, and the edges of the side curvature sealed by sealing gel (12).

2. The flexible supine wearable pelvic floor drug nebulization device of claim 1, wherein: the waist tightening belt (11) further comprises an adjustable adhesive strip (15).
During use, the nebulization device is worn on the lower part of the body, with the waist tightening belt (11) used to secure the wearable nebulization hood (1) around the waist, and the adjustable adhesive strip (15) being used for fixation and adjustment.
The adjustable adhesive strip (15) comprises a first adjustable adhesive strip (151) and a second adjustable adhesive strip (152), with the first and second adjustable adhesive strips (151, 152) located on the left and right sides of the waist, respectively.

3. The flexible supine wearable pelvic floor drug nebulization device of claim 1, wherein: the nebulization port (17) is located at the bottom of the wearable nebulization hood (1) and directly below the expandable nebulization membrane (13).

4. The flexible supine wearable pelvic floor drug nebulization device of claim 1, further comprising: a drainage bag (3).
The drainage bag (3) being connected to the drainage port (14) of the wearable nebulization hood (1).

5. The flexible supine wearable pelvic floor drug nebulization device of claim 4, wherein: the drainage port (14) is connected to the inlet of the drainage bag (3), and the drainage port (14) is located at the bottom of the wearable nebulization hood (1), for discharging the residual medicinal solution condensed from the mist after nebulization.

6. The flexible supine wearable pelvic floor drug nebulization device of claim 1, wherein: the expandable nebulization membrane (13) is made of a film material, and the nebulized mist is blown into the expandable nebulization membrane (13) through the nebulization port (17), forming a positive pressure inside the expandable nebulization membrane (13) to expand it, adapting to the shape of the buttocks and legs for comfortable wear.

7. The flexible supine wearable pelvic floor drug nebulization device of claim 1, wherein: the sealing gel (12) is made of a silicone gel material with antibacterial, waterproof, leak-proof, soft, and skin-friendly properties.

8. The flexible supine wearable pelvic floor drug nebulization device of claim 1, further comprising: a spraying device. The spraying device being connected to the nebulization port (17) to clean the anal area.

9. The flexible supine wearable pelvic floor drug nebulization device of claim 1, wherein: the nebulizer (2) is a compressed air nebulizer or a micro-mesh ultrasonic nebulizer, and the output port of the nebulizer (2) is connected to the nebulization port (17) of the wearable nebulization hood (1) via a conduit.

10. The flexible supine wearable pelvic floor drug nebulization device of claim 1, wherein: the exhaust port (16) is a one-way exhaust port.
